(19) United States
(12) Patent Application Publication
(10) Pub. No.: US 2025/0134408 A1
(43) Pub. Date: May 1, 2025

(54) Determining Respiration Rates Based On Audio Streams and User Conditions

(71) Applicant: Apple Inc., Cupertino, CA (US)

(72) Inventors: Juri Minxha, Seattle, WA (US); Narimene Lezzoum, San Jose, CA (US); Vikramjit Mitra, Pleasanton, CA (US); Erdrin Azemi, San Mateo, CA (US)

(21) Appl. No.: 18/901,467

(22) Filed: Sep. 30, 2024

Related U.S. Application Data
(60) Provisional application No. 63/593,186, filed on Oct. 25, 2023.

(51) Int. Cl. A61B 5/08

(52) U.S. Cl. CPC A61B 5/0816

(57) ABSTRACT

A system can receive an input indicating a user condition. The system can also receive an internal audio stream from an in-ear microphone and an external audio stream from an external microphone of a head worn system. The system can determine a respiration rate of a user based on the internal audio stream, the external audio stream, and the input indicating the user condition. In some implementations, the respiration rate may be determined from a respiration signal in the internal audio stream and/or the external audio stream. The respiration signal may measure breathing of the user. In some implementations, the system can invoke a machine learning model to determine the respiration signal from the internal audio stream and/or the external audio stream based on the user condition.

### RELATED APPLICATIONS

This patent application claims the benefit of priority of U.S. Provisional Application No. 63/593,186, filed Oct. 25, 2023, which is incorporated herein by reference in its entirety.

### BACKGROUND

### Field

This disclosure relates generally to health monitoring and, more specifically, to determining respiration rates of users based on audio streams and user conditions. Other aspects are also described.

### Background Information

A respiration rate is a measure of a person's breathing and may be calculated as a number of breaths per unit of time. A respiration cycle may include an inhalation and an exhalation performed by the person. A person's respiration rate may increase with exercise and other activity and may decrease with rest. A person's respiration rate may be one of several measurements that may be taken to determine a person's health and activity.

### SUMMARY

Implementations of this disclosure include utilizing input that provides situational awareness to a system to enable the system to determine a user's respiration rate based on different utilizations of internal and/or external audio streams. Some implementations may include a respiration detection system configured to receive an input indicating a user condition, an internal audio stream from an in-ear microphone of a head worn system, and an external audio stream from an external microphone of the head worn system. For example, the head worn system could comprise an earbud including the in-ear microphone and the external microphone. In another example, the head worn system could comprise an earbud including the in-ear microphone and a headset or eyewear including the external microphone. The in-ear microphone may be configured to pick up sound in a cavity of an ear of the user and the external microphone may be configured to pick up sound outside of the head worn system. The input may comprise user input and/or sensor input for indicating the user condition. For example, the input could come from a companion device, such as a smartphone, smartwatch, wearable device, the head worn system, or another mobile device utilized by the user. The user input may include the user indicating the user condition, such as the user indicating a type of exercise being performed (e.g., push-ups or running) or an indication of activity, such as walking, meditating, eating, drinking, or resting by the user. The sensor input may include an indication of the user condition via one or more sensors indicating a vital sign of the user (e.g., heart rate, temperature), movement of the user (e.g., a repetition, such as walking, running, jumping, or lifting), or location of the user (e.g., indoors or outdoors).

The respiration detection system can determine a respiration rate of the user based on the internal audio stream, the external audio stream, and the input indicating the user condition. For example, the respiration rate may be determined from one or more respiration signals, that measure breathing of the user, from the internal audio stream and/or the external audio stream, according to the user condition. In some implementations, the one or more respiration signals may be weighted to measure breathing of the user for the respiration rate. In some implementations, the respiration detection system can invoke a machine learning model to determine the respiration signal from the internal audio stream and/or the external audio stream. The machine learning model can determine the respiration signal based on the user condition indicating utilizations of the internal audio stream and the external audio stream based on the user condition. The respiration detection system can determine the respiration rate of the user based on the respiration signal. Other aspects are also described and claimed.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 is an example of a respiration detection system for determining respiration rates of users based on audio streams and user conditions.

FIG. 2 is an example of an internal audio stream from an in-ear microphone and an external audio stream from an external microphone received during mediation.

FIG. 3 is an example of an internal audio stream from an in-ear microphone and an external audio stream from an external microphone received during exercise.

FIG. 4 is an example of an enhancement system for enhancing audio streams for determining respiration rates of users.

FIG. 5 is a flowchart of an example of a process for determining respiration rates of users based on audio streams and user conditions.

### DETAILED DESCRIPTION

An audio stream from a microphone may be utilized to determine a respiration rate of a user. For example, a user might wear an earbud with a microphone that generates an audio stream. The audio stream may include a respiration signal from which a respiration rate can be calculated. For example, the respiration signal could comprise a respiration-aligned spectral signature associated with breathing of the user, e.g., cycles of inhalation and exhalation.

In some situations, an internal audio stream from an in-ear microphone may provide a stronger respiration signal for determining a respiration rate than an external audio stream from an external microphone. For example, during meditation, an in-car microphone may be better situated to detect breathing of the user. In other situations, the external audio stream from the external microphone may be better situated to detect breathing of the user. For example, during exercise, the in-ear microphone may detect more aggressor signals, such as sounds from the user running on a treadmill causing the internal audio stream to be distorted. As a result, the external microphone be better situated to detect breathing of the user. What is needed is a system that can reliably determine a respiration rate of a user.

Implementations of this disclosure address problems such as these by utilizing input that provides situational awareness to a system to enable the system to determine a user's respiration rate based on different utilizations of internal and/or external audio streams. Some implementations may include a respiration detection system configured to receive an input indicating a user condition, an internal audio stream from an in-ear microphone of a head worn system, and an external audio stream from an external microphone of the head worn system. For example, the head worn system could comprise an earbud including the in-ear microphone and the external microphone. In another example, the head worn system could comprise an earbud including the in-ear microphone and a headset or eyewear including the external microphone. The in-car microphone may be configured to pick up sound in a cavity of an car of the user, and the external microphone may be configured to pick up sound outside of the head worn system. The input may comprise user input and/or sensor input for indicating the user condition. For example, the input could come from a companion device, such as a smartphone, smartwatch, wearable device, the head worn system, or another mobile device utilized by the user. The user input may include the user indicating the user condition, such as the user indicating a type of exercise being performed (e.g., push-ups or running) or an activity, such as walking, meditating, eating, drinking, or resting by the user. The sensor input may include an indication of the user condition via one or more sensors indicating a vital sign of the user (e.g., heart rate, temperature), movement of the user (e.g., a repetition, such as walking, running, jumping, or lifting), or location of the user (e.g., indoors or outdoors).

The respiration detection system can determine a respiration rate of the user based on the internal audio stream, the external audio stream, and the input indicating the user condition. For example, the respiration rate may be determined from one or more respiration signals, that measure breathing of the user, from the internal audio stream and/or the external audio stream, according to the user condition. In some implementations, the one or more respiration signals may be weighted to measure breathing of the user for the respiration rate. In some implementations, the respiration detection system can invoke a machine learning model to determine the respiration signal from the internal audio stream and/or the external audio stream. The machine learning model can determine the respiration signal based on the user condition indicating utilizations of the internal audio stream and the external audio stream based on the user condition. The respiration detection system can determine the respiration rate of the user based on the respiration signal. As a result, the respiration detection system can reliably determine a respiration rate of a user in many different situations.

In some implementations, to detect respiration of a user (e.g., rate and/or phase), the respiration detection system can utilize audio streams from both inward and outward facing microphones on earbuds. This may enable the respiration detection system to resolve a wider array of aggressor signals associated with sounds (e.g., music, phone, speech, and other environmental noise) that may differentially affect each of the microphones. For example, the respiration detection system may utilize blind source separation on these two channels (e.g., from the inward and outward facing microphones) to extract the spectral signature of respiration from the noise.

In some implementations, using both audio streams can provide situational awareness to discern between (1) high noise, more distorted sensor data, and (2) low noise, better sensor signal clarity conditions. Using multiple audio channels may enable disentangling a source signal from aggressor signals to generate a respiration-relevant signal. In some cases, the system may enhance a respiration signal using blind source separation to reduce the role of the aggressor signals, such as background noise (e.g., speech and music), and/or aggressor signals generated by the earbuds (e.g., music or speech from an earbud speaker). Reducing such aggressor signals may improve the audio quality of the restoration audio sensor data to enable an accurate estimation of respiration rate and other respiration associated metrics.

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

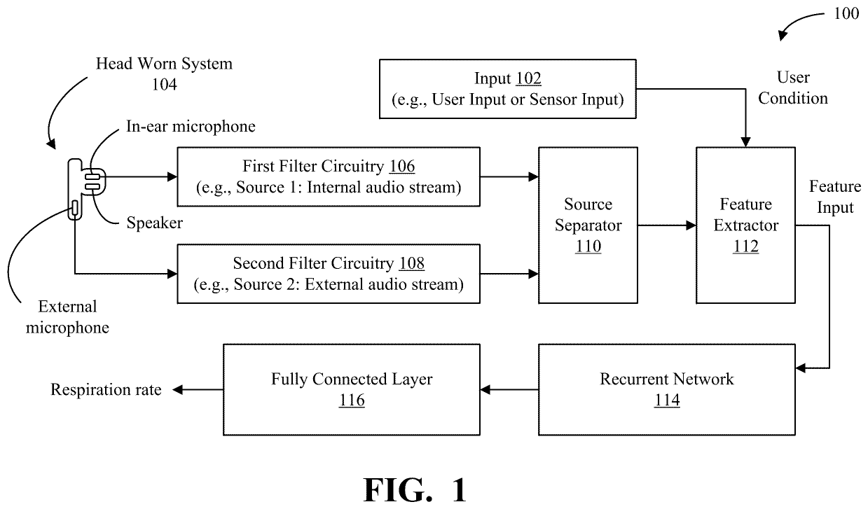

FIG. 1 is an example of a respiration detection system 100 for determining respiration rates of users based on audio streams and user conditions. In various implementations, the respiration detection system 100 can receive input 102 that provides situational awareness to enable the respiration detection system 100 to determine a person's respiration rate based on different utilizations of an internal audio stream and an external audio stream. The respiration detection system 100 may include a head worn system 104, input circuitry, and a machine learning model. For example, the head worn system 104 may comprise earbuds, goggles, or glasses, which could also include virtual reality (VR), augmented reality (AR), or mixed reality. Further, the input circuitry may include first filter circuitry 106, second filter circuitry 108, a source separator 110, and a feature extractor 112. In another example, the machine learning model may include a recurrent network 114 and a fully connected layer 116.

The head worn system 104 may include an in-ear microphone and an external microphone. The in-car microphone can generate an internal audio stream. For example, the in-car microphone may be configured to pick up sound in a cavity of an car of the user and generate the internal audio stream based on the sound. Additionally, the external microphone can generate an external audio stream. For example, the external microphone may be configured to pick up sound outside of the head worn system 104 and generate the external audio stream based on the sound. In some cases, the head worn system 104 may also include a speaker for playing audio data, such as music, sound from a phone call, and/or noise cancellation. In one example, the head worn system 104 may comprise an earbud that includes the in-ear microphone, the external microphone, and/or the speaker. In another example, the head worn system 104 may comprise an earbud that includes the in-ear microphone and a headset or eyewear that includes the external microphone. In another example, the head worn system 104 may include a pair of earbuds, each including an in-ear microphone, an external microphone, and/or a speaker.

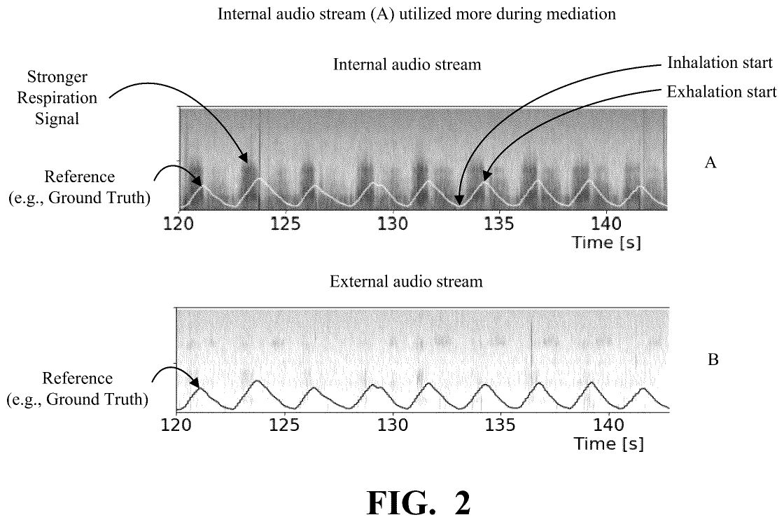

The input circuitry may receive the internal audio stream and the external audio stream as sources in separate channels. For example, the first filter circuitry 106 may receive the internal audio stream as a first source in a first channel and the second filter circuitry 108 may receive the external audio stream as a second source in a second channel. The first filter circuitry 106, and the second filter circuitry 108, may each include analog and/or digital circuitry to perform low pass filtering to generate temporal spectral or time-frequency representations of the audio data (sounds) picked up by the respective microphone in an audible encompassing range, e.g., 0 to 2 kHz. For example, the first filter circuitry 106 may include low pass filtering to generate time-frequency representations of sounds from the internal audio stream, and the second filter circuitry 108 may include low pass filtering to generate time-frequency representations of sounds from the external audio stream. For example, with additional reference to FIG. 2, a time-frequency representation of an internal audio stream (graph “A”) and a time-frequency representation of an external audio stream (graph “B”) is shown.

The source separator 110 may receive the time-frequency representations of sounds from the internal audio stream and the external audio stream in the separate channels. In some implementations, the source separator 110 may perform additional signal conditioning, such as amplifying the time-frequency representations in the separate channels. The feature extractor 112 may receive the time-frequency representations in the separate channels from the source separator 110. The feature extractor 112 may perform digital signal processing (DSP) to extract features from the internal audio stream and/or the external audio stream based on the time-frequency representations. For example, the feature extractor 112 may utilize a DSP algorithm, such as Mel filter bank energy (MFE), Mel frequency cepstral coefficients (MFCC), or spectrogram, according to various parameters specifying, for example, frame length, frame stride, frequency bands, normalization, and/or noise floor. The feature extractor 112 may extract the features from the time-frequency representations for the machine learning model to generate inferences.

The machine learning model may receive the features from the feature extractor 112. The machine learning model may, for example, be or include one or more of a neural network (e.g., a gated recurrent network, multilayer perceptron, convolutional neural network, recurrent neural network, deep neural network, or other neural network), decision tree, vector machine, Bayesian network, cluster-based system, genetic algorithm, deep learning system separate from a neural network, or other machine learning model. For example, the machine learning model may include the recurrent network 114 to receive the features from the feature extractor 112. The recurrent network 114 may implement a gated recurrent network (e.g., a 2 layer GRU) with a bi-directional flow that enables the output from one or more nodes to affect a subsequent input to the same nodes (e.g., the recurrent network 114 may utilize an internal state to process sequences of inputs). The machine learning model may also include the fully connected layer 116 to receive an output from the recurrent network 114. For example, the fully connected layer 116 could comprise a multilayer perceptron that implements a feed-forward neural network that learns features to generate inferences.

The input 102 may comprise user input (e.g., from the user) and/or sensor input (e.g., from one or more sensors) for indicating the user condition. For example, the input 102 could be provided via the head worn system 104 or another device, such as a companion device, (e.g., a smartphone, smartwatch, wearable device, or other mobile device utilized by the user). The user input may include the user directly indicating the user condition, such as by the user inputting a type of exercise being performed (e.g., push-ups or running) or an activity, such as walking, meditating, eating, drinking, or resting. For example, the user may input information into the device to indicate the user condition, such as by typing the type of exercise or activity, or selecting an icon associated with the type of exercise or the activity, via an application program running on the device. The sensor input may indicate the user condition via a measurement from one or more sensors of the device. The measurement may indicate a vital sign of the user (e.g., heart rate, temperature), movement of the user (e.g., a repetition, such as walking, running, jumping, or lifting), or location of the user (e.g., indoors or outdoors, or within a geofence). For example, the one or more sensors may include one or more gyroscopes, accelerometers, inertial measurement units (IMU), proximity sensors, ambient light sensors, location sensors (e.g., global positioning system and/or Wi-Fi), pedometers, heart rate sensors, and/or blood pressure sensors. The sensor input may enable the user to indirectly indicate the user condition, such as by the one or more sensors sensing a type of exercise performed by the user (e.g., push-ups or running) or an indication of activity, such as walking, meditating, eating, drinking, or resting by the user. For example, the one or more sensors may enable an application program running on the device to sense that the user is performing the type of exercise or the activity.

The respiration detection system 100 can determine a respiration rate (and/or phase) of the user, such as a number of breaths per unit of time, each breath including an inhale and an exhale, based on the internal audio stream, the external audio stream, and the input 102 indicating the user condition. The respiration rate may result from an output generated by the machine learning model based on inputs through the input circuitry. For example, the input circuitry, such as the feature extractor 112, may receive the input 102 indicating the user condition. The input 102 may cause the feature extractor 112 to extract features from the audio streams in different ways depending on the user condition that is indicated. In some cases, the user condition may cause the respiration detection system 100 (e.g., via the machine learning model receiving the feature input) to give greater weight to one of the audio streams (e.g., the internal audio stream) and lesser weight to the other of the audio streams (e.g., the external audio stream). For example, the respiration detection system 100 may apply greater weight to the internal audio stream when aggressor signals in the internal audio stream are below a threshold (e.g., indicated by a user condition associated with meditation). In another example, the respiration detection system 100 may apply greater weight to the external audio stream when there are more aggressor signals in the internal audio stream than the external audio stream (e.g., indicated by a user condition associated with running). In other cases, the user condition may cause one of the audio streams (e.g., the internal audio stream) to be gated (e.g., blocked) and the other of the audio streams (e.g., the external audio stream) to pass to the machine learning model. Thus, the respiration rate may be determined from one or more respiration signals, that measure breathing of the user in one or more streams, according to the user condition. As a result, the respiration detection system 100 can reliably determine a respiration rate of a user in many different situations corresponding to the user conditions.

By way of example, with additional reference to the example in FIG. 2, the respiration detection system 100 can receive an internal audio stream (graph “A”) from the in-car microphone and an external audio stream (graph “B”) from the external microphone. The graphs A and B are time-frequency spectral analysis plots, where time is along the x-axis and spectral content or frequency is in the y-axis, as shown. The “bright” portion of each column is the frequency band in which the signal is strongest and therefore more easily detectable. It can be seen that these strong frequency bands repeat over time. The occurrence of a strong frequency band is between the end of an inhalation phase and the start of a next exhalation phase (as marked in FIG. 2.) The strong frequency bands are more visible in graph A than in graph B. A reference (e.g., ground truth) plot is overlayed on graphs A and B as shown, which indicates actual respiration of the user that was measured using additional equipment (e.g., a sensor belt worn by the user).

Note that the audio streams shown in FIG. 2 are during a first period corresponding to mediation. The mediation may be a user condition indicated by the input 102. For example, the mediation may be indicated by user input (e.g., the user selecting meditation in an application program) and/or by sensor input (e.g., one or more sensors indicating less user movement during the first period).

During the meditation, there may be fewer aggressor signals (e.g., music, phone, speech, and other environmental noise) in either audio stream. For example, the aggressor signals in the internal audio stream may be below a threshold. As a result, the respiration detection system 100 can utilize the internal audio stream more (e.g., giving greater weight and/or passing that signal) and the external audio stream less (e.g., giving lesser weight and/or gating that signal) to determine a respiration signal from which a respiration rate can be calculated. For example, the respiration detection system 100 can better distinguish the respiration signal from aggressor signals in the internal audio stream. Thus, the respiration detection system 100 utilizes more of the internal audio stream, associated with a stronger respiration signal, based on the user condition that is determined in the first period.

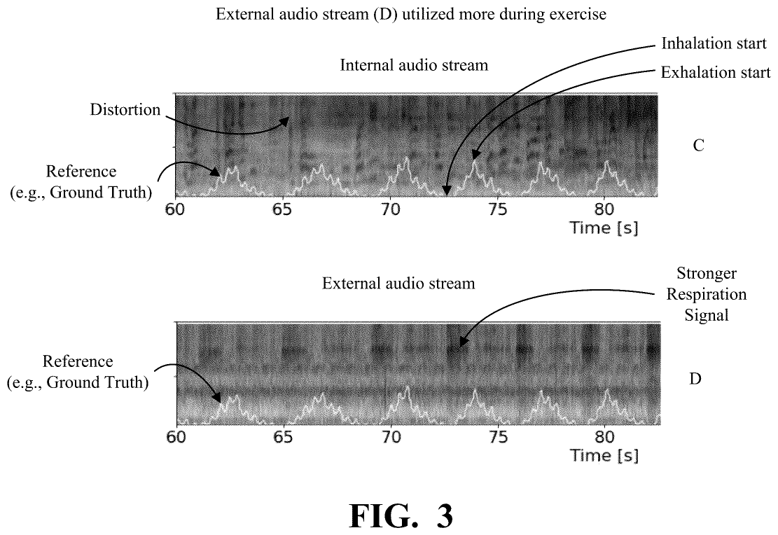

In another example, with additional reference to the example in FIG. 3, the respiration detection system 100 can receive the internal audio stream (graph “C”) from the in-car microphone and the external audio stream (graph “D”) from the external microphone. The audio streams shown in FIG. 3 are during a second period corresponding to a type of exercise (e.g., running). Like the graphs A and B of FIG. 2, the graphs C and D of FIG. 3 are time-frequency spectral analysis plots, where time is along the x-axis and spectral content or frequency is in the y-axis, as shown. The “bright” portion of each column is the frequency band in which signals are detected (e.g., respiration or aggressor signals). It can be seen that these frequency bands include distortion, making detection of the respiration signal in the internal audio stream more difficult (e.g., a strong frequency band between the end of an inhalation phase and the start of a next exhalation phase, as marked in FIG. 3). The strong frequency bands are clearer in graph D than in graph C. A reference (e.g., ground truth) plot is overlayed on each graphs C and D as shown, which indicates actual respiration of the user that was measured using additional equipment (e.g., the sensor belt worn by the user).

Note that the audio streams shown in FIG. 3 are during the second period corresponding to the type of exercise (e.g., running). The exercise may be a new user condition indicated by the input 102. For example, the running may be indicated by user input (e.g., the user selecting a run in an application program) and/or by sensor input (e.g., one or more sensors indicating faster movement and/or bouncing during the second period).

During the exercise, the internal audio stream may include greater distortion caused by aggressor signals, such as sounds from the user running on a treadmill, rendering the respiration signal from the internal audio stream weaker (e.g., more difficult to distinguish). For example, there may be more aggressor signals in the internal audio stream than the external audio stream. As a result, the respiration detection system 100 can utilize the internal audio stream less (e.g., less greater weight and/or gating that signal) and the external audio stream more (e.g., giving more weight and/or passing that signal) to determine a respiration signal from which a respiration rate can be calculated. For example, the respiration detection system 100 can better distinguish the respiration signal from aggressor signals in the external audio stream. Thus, the respiration detection system 100 utilizes more of the external audio stream, again associated with a stronger respiration signal, based on the new user condition that is determined in the second period. Additionally, the respiration detection system 100 can reliably determine the respiration rate of the user in many different situations.

In some implementations, the respiration detection system 100 may trigger a new determination of the respiration rate based on the input 102. For example, when the input 102 may indicate a new user condition, such as the user performing a new type of exercise (e.g., push-ups, instead of running) or a new activity (e.g., walking, eating, or drinking, instead of meditating). The respiration detection system 100 may then automatically trigger an update to the determination of the respiration rate based on the new user condition.

In some implementations, the machine learning model may comprise separate machine learning models in separate channels. For example, the machine learning model may comprise a first machine learning model that determines a first respiration signal from features extracted from the internal audio stream and a second machine learning model that determines a second respiration signal from features extracted from the external audio stream. For example, the first machine learning model may include a recurrent network and a fully connected layer in a first channel, and the second machine learning model may include another recurrent network and another fully connected layer in a second channel. The respiration detection system 100 may determine the respiration rate based on utilizations of the first respiration signal for determining a first respiration rate and the second respiration signal for determining a second respiration rate. In some cases, the respiration detection system 100 may utilize ensemble learning based on the machine learning models in the separate channels.

In some implementations, the respiration detection system 100 may include additional audio streams from additional microphones (e.g., additional sources in additional channels). For example, when the head worn system 104 comprises a pair of earbuds, each earbud may generate an internal audio stream and an external audio stream that is received in separate channels by the input circuitry then the machine learning model as described herein. In another example, when the head worn system 104 comprises a companion device, such as a smartphone, smartwatch, headset, eyewear, wearable device, or other mobile device utilized by the user, that device may also generate an additional audio stream from an additional microphone (e.g., another external microphone, generating another external audio stream). That additional audio stream may also be received in a separate channel by the input circuitry then the machine learning model as described herein.

In some implementations, the respiration detection system 100 may include post-processing that utilizes the internal audio stream, the external audio stream, additional audio streams, and/or the input 102. In some cases, the post-processing may be utilized to validate the respiration rate of the user. For example, the respiration detection system 100 may utilize an additional audio stream, and/or sensor input, to validate that a determined respiration signal, aggressor signal, and/or respiration rate is consistent with a user condition (e.g., sensing an elevated heart rate as consistent with running, as opposed to meditation). In some cases, the post-processing may determine a trust score associated with the respiration rate based on the user condition. For example, the post-processing may utilize the trust score to determine whether the respiration rate should be reported to the user (e.g., via the application program running on the device) or measured again. In some cases, reporting the respiration rate to the user may require the trust score to exceed a threshold.

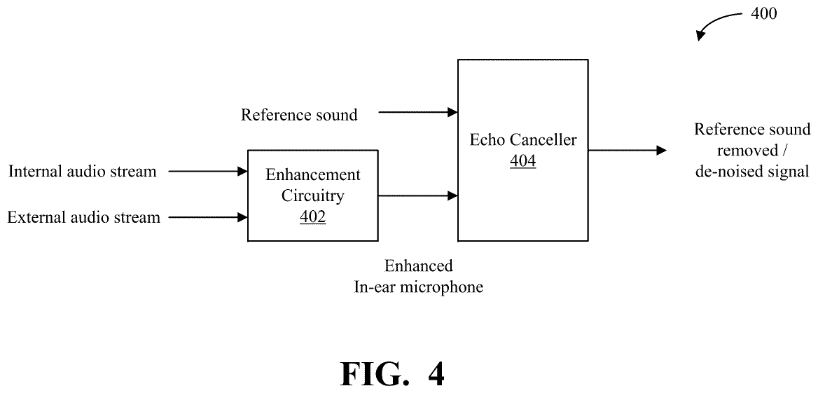

FIG. 4 is an example of an enhancement system 400 for enhancing audio streams for determining respiration rates of users. In some implementations, the respiration detection system 100 may include the enhancement system 400. The enhancement system 400 may include enhancement circuitry 402 and/or an echo canceller 404. The enhancement circuitry 402 may receive the internal audio stream and the external audio stream as sources in separate channels. In some implementations, the enhancement circuitry 402 may be implemented between the filter circuitry of FIG. 1 (e.g., the first filter circuitry 106 and the second filter circuitry 108) and the source separator 110. The enhancement circuitry 402 may include conditioning circuitry to enhance the internal audio stream, based on the external audio stream, to generate an enhanced audio stream. For example, the enhancement circuitry 402 may utilize superposition, based on the external audio stream, to enhance the internal audio stream. The enhanced audio stream may then be used in the respiration detection system 100 as a replacement stream (e.g., a replacement for the internal audio stream). In some implementations, the enhancement circuitry 402 may include conditioning circuitry to enhance the external audio stream, based on the internal audio stream, to generate the enhanced audio stream.

The respiration rate may be determined from a respiration signal in the enhanced audio stream. In some cases, further conditioning may be performed by the echo canceller 404. For example, the echo canceller 404 may receive the enhanced audio stream and a reference sound. The reference sound may correspond to a sound that is played by the speaker of the head worn system 104, such as music, sound from a phone call, and/or noise cancellation. The echo canceller 404 may include cancellation circuitry to remove the reference sound from the enhanced audio stream (e.g., canceling the sound playing by the speaker) to generate a de-noised respiration signal, free from the reference sound, from which the respiration rate can be determined.

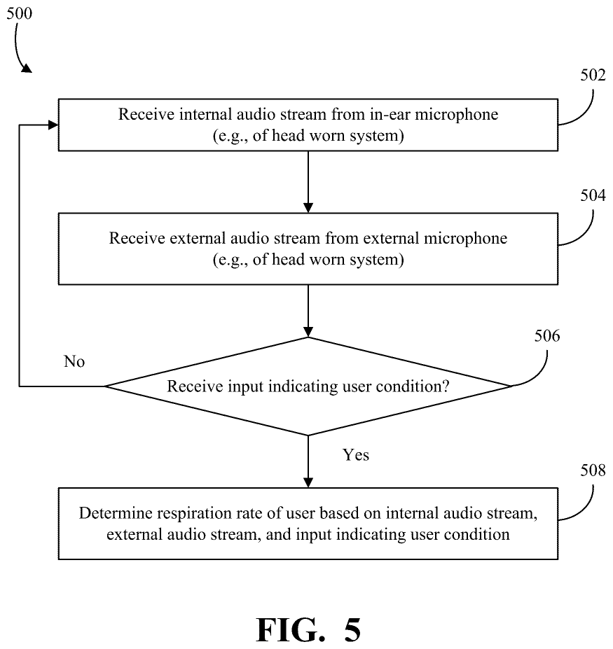

To further describe some implementations in greater detail, reference is next made to examples of processes which may be performed by or using a system for determining respiration rates of users based on audio streams and user conditions. FIG. 5 is a flowchart of an example of a process 500 for determining respiration rates. The process 500 can be executed using computing devices, such as the systems, hardware, and software described with respect to FIGS. 1-4. The process 500 can be performed, for example, by executing a machine-readable program or other computer-executable instructions, such as routines, instructions, programs, or other code. The steps, or operations, of the process 500 or another process, method, technique, or algorithm described in connection with the implementations disclosed herein can be implemented directly in hardware, firmware, software executed by hardware, circuitry, or a combination thereof.

For simplicity of explanation, the process 500 is depicted and described herein as a series of steps or operations. However, the steps or operations in accordance with this disclosure can occur in various orders and/or concurrently. Additionally, other steps or operations not presented and described herein may be used. Furthermore, not all illustrated steps or operations may be required to implement a process in accordance with the disclosed subject matter.

At operation 502, a system (e.g., the respiration detection system 100) may receive an internal audio stream from an in-ear microphone of a head worn system (e.g., of the head worn system 104). For example, the head worn system could comprise an earbud including the in-car microphone. The in-car microphone may be configured to pick up sound in a cavity of an car of a user.

At operation 504, the system may receive an external audio stream from an external microphone (e.g., of the head worn system). For example, the head worn system could comprise an earbud including the external microphone. In another example, the head worn system could comprise a headset or eyewear including the external microphone. The external microphone may be configured to pick up sound outside of the head worn system. In some implementations, the internal audio stream may be enhanced by the external audio stream to generate an enhanced audio stream. In some implementations, the external audio stream may be enhanced by the internal audio stream to generate an enhanced audio stream. In some implementations, an echo canceller may cancel sound played by a speaker of the head worn device in the enhanced audio stream.

At operation 506, the system may determine whether an input (e.g., the input 102) indicating a user condition is received. The input may comprise user input from the user, sensor input from one or more sensors, or a combination thereof, for indicating the user condition. For example, the input could come from a companion device, such as a smartphone, smartwatch, wearable device, the head worn system, or another mobile device utilized by the user. The user input may include the user indicating the user condition, such as the user indicating a type of exercise being performed by the user (e.g., push-ups or running) or an indication of an activity being performed, such as walking, meditating, eating, drinking, or resting by the user. The sensor input may include indicating the user condition via one or more sensors indicating a vital sign of the user (e.g., heart rate, temperature), movement of the user (e.g., a repetition, such as walking, running, jumping, or lifting), or location of the user (e.g., indoors or outdoors).

If an input indicating a user condition is not received (“No”), the system may return to operation 502, and operation 504, to continue receiving the internal audio stream and the external audio stream, respectively, and monitoring for the input. If the input is received (“yes”), at operation 508, the system may determine a respiration rate of the user based on the internal audio stream, the external audio stream, and the input indicating the user condition. For example, the respiration rate may be determined from one or more respiration signals, that measure breathing of the user, from the internal audio stream and/or the external audio stream, according to the user condition. In some implementations, the one or more respiration signals may be weighted to measure breathing of the user for the respiration rate. In some implementations, the respiration detection system can invoke a machine learning model to determine the respiration signal from the internal audio stream and/or the external audio stream. The machine learning model can determine the respiration signal based on the user condition indicating utilizations of the internal audio stream and the external audio stream based on the user condition. The machine learning model could be run by one or more processors of a system including the in-ear microphone and/or the external microphone, such as the head worn system, or run by a companion device in communication with the system including the in-car microphone and/or the external microphone. For example, the machine learning model could be run by a smartphone, a smartwatch, a tablet, or the head worn system, which could comprise earbuds, goggles, or glasses (which could also include VR, AR, or mixed reality). The respiration detection system can determine the respiration rate of the user based on the respiration signal. For example, the machine learning model may determine the respiration rate based on the respiration signal. In some implementations, the system can store the respiration rate on a companion device in communication with the in-car microphone and/or the external microphone. For example, the respiration rate could be streamed to the companion device and stored in health records of the user maintained on the companion device.

In some implementations, the system can return to operation 502 to continue receiving the internal audio stream and the external audio stream, respectively, and monitoring for a new input indicating a user condition. In some implementations, the system can return to operation 502 to periodically update the respiration rate (e.g., the respiration rate may be updated periodically, such as every N minutes, where N is an integer value). Thus, the process 500 may reduce the effect of background aggressors (e.g., background noise, speech, music) to enhance the audio signal relevant to breathing and improve estimation of relevant parameters such as respiration rate. As a result, the respiration detection system can reliably determine a respiration rate of a user in many different situations.

In some implementations, a method may include receiving an input indicating a user condition; receiving an internal audio stream from an in-ear microphone and an external audio stream from an external microphone of a head worn system; and determining a respiration rate of a user based on the internal audio stream, the external audio stream, and the input indicating the user condition. The in-ear microphone may be configured to pick up sound in a cavity of an car of the user and the external microphone may be configured to pick up sound outside of the head worn system. The method may include triggering determination of the respiration rate when the user condition indicates the user is exercising. The respiration rate may be determined from a respiration signal that measures breathing of the user, and the method may include invoking a machine learning model to determine the respiration signal from at least one of the internal audio stream or the external audio stream based on the user condition. In some cases, a greater weight may be applied to either the internal audio stream or the external audio stream based on the user condition. In some cases, a greater weight is applied to the internal audio stream when aggressor signals in the internal audio stream are below a threshold. In some cases, a greater weight is applied to the external audio stream when there are more aggressor signals in the internal audio stream than the external audio stream. In some cases, the input may include a measurement from a sensor indicating a vital sign or movement of the user. In some cases, the input may include an indication of a location of the user being indoors or outdoors. In some cases, the input may include an indication of a type of exercise performed by the user. In some cases, the input may include an indication of whether the user is walking, meditating, eating, drinking, or resting. In some cases, the input may be from a wearable device of the user. In some cases, the respiration rate may be determined from a respiration signal by distinguishing the respiration signal from aggressor signals caused by ambient sounds outside of the head worn system. In some cases, the respiration rate may be determined based on a machine learning model determining a respiration signal from features extracted from the internal audio stream and the external audio stream. In some cases, the respiration rate may be determined based on a first machine learning model that determines a first respiration signal from features extracted from the internal audio stream and a second machine learning model that determines a second respiration signal from features extracted from the external audio stream. In some cases, the head worn system comprises a first earbud, including the in-car microphone and the external microphone, and a second earbud. In some cases, the head worn system may include an earbud, including the in-ear microphone, and a headset or eyewear including the external microphone. The method may include validating the respiration rate of the user based on an additional audio stream from an additional microphone. The method may include determining a trust score associated with the respiration rate based on the user condition. The method may include enhancing the internal audio stream, based on the external audio stream, to generate an enhanced audio stream, wherein the respiration rate is determined from a respiration signal in the enhanced audio stream.

In some implementations, a non-transitory computer readable medium can store instructions operable to cause one or more processors to perform operations comprising receiving an internal audio stream from an in-ear microphone and an external audio stream from an external microphone of a head worn system; invoking a machine learning model to determine a respiration signal that measures breathing of a user, the machine learning model determining the respiration signal based on a user condition indicating utilizations of the internal audio stream and the external audio stream; and determining a respiration rate of the user based on the respiration signal. The machine learning model can give greater weight to one of the internal audio stream or the external audio stream, and lesser weight to the other of the internal audio stream or the external audio stream, based on the user condition. The operations may include selecting between either the internal audio stream or the external audio stream based on the user condition. The operations may include gating one of the internal audio stream or the external audio stream based on the user condition so that the machine learning model determines the respiration signal based on the other of the internal audio stream or the external audio stream. The operations may include canceling a sound, playing by a speaker of the head worn system, from the internal audio stream or the external audio stream. The operations may include receiving an input, from a headset or eyewear, indicating the user condition. The machine learning model may comprise a recurrent network and a fully connected layer. The operations may include validating the respiration signal based on an additional audio stream from an additional microphone. The operations may include validating an aggressor signal, caused by an ambient sound outside of the head worn system, based on an additional audio stream from an additional microphone. The operations may include determining a trust score associated with the respiration rate based on the user condition.

In some implementations, a non-transitory computer readable medium can store instructions operable to cause one or more processors to perform operations comprising receiving an input indicating a user condition; receiving an internal audio stream from an in-car microphone and an external audio stream from an external microphone of a head worn system; and determining a respiration rate of a user based on the internal audio stream, the external audio stream, and the input indicating the user condition. The operations may include storing the respiration rate on a companion device in communication with at least one of the in-ear microphone or the external microphone. The respiration rate may be updated periodically. The operations may include invoking a machine learning model to determine the respiration rate, wherein the machine learning model is run by a system including at least one of the in-car microphone or the external microphone. The operations may include invoking a machine learning model to determine the respiration rate, wherein the machine learning model is run by a companion device in communication with at least one of the in-ear microphone or the external microphone.

As used herein, the term “circuitry” refers to an arrangement of electronic components (e.g., transistors, resistors, capacitors, and/or inductors) that is structured to implement one or more functions. For example, a circuit may include one or more transistors interconnected to form logic gates that collectively implement a logical function.

It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

As described above, one aspect of the present technology is the gathering and use of data available from specific and legitimate sources for determining respiration rates of users based on audio streams and user conditions. The present disclosure contemplates that in some instances, this gathered data may include personal information data that uniquely identifies or can be used to identify a specific person. Such personal information data can include demographic data, location-based data, online identifiers, telephone numbers, email addresses, home addresses, data or records relating to a user's health or level of fitness (e.g., vital signs measurements, medication information, exercise information), date of birth, or any other personal information.

The present disclosure recognizes that the use of such personal information data, in the present technology, can be used to the benefit of users. For example, the personal information data can be used to determine respiration rates of users. For instance, health and fitness data may be used, in accordance with the user's preferences to provide insights into their general wellness or may be used as positive feedback to individuals using technology to pursue wellness goals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominent and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations that may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, such as in the case of determining respiration rates of users based on audio streams and user conditions, the present technology can be configured to allow users to select to “opt in” or “opt out” of participation in the collection of personal information data during registration for services or anytime thereafter.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level), controlling how data is stored (e.g., aggregating data across users), and/or other methods such as differential privacy.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users based on aggregated non-personal information data or a bare minimum amount of personal information, such as the content being handled only on the user's device or other non-personal information available to the content delivery services.

In utilizing the various aspects of the embodiments, it would become apparent to one skilled in the art that combinations or variations of the above embodiments are possible for determining respiration rates of users based on audio streams and user conditions. Although the embodiments have been described in language specific to structural features and/or methodological acts, it is to be understood that the appended claims are not necessarily limited to the specific features or acts described. The specific features and acts disclosed are instead to be understood as embodiments of the claims useful for illustration.

What is claimed is:

1. A method, comprising:
receiving an input indicating a user condition;
receiving an internal audio stream from an in-ear microphone and an external audio stream from an external microphone of a head worn system; and
determining a respiration rate of a user based on the internal audio stream, the external audio stream, and the input indicating the user condition.

2. The method of claim 1, further comprising:
triggering determination of the respiration rate when the user condition indicates the user is exercising.

3. The method of claim 1, wherein the respiration rate is determined from a respiration signal that measures breathing of the user, and further comprising:
invoking a machine learning model to determine the respiration signal from at least one of the internal audio stream or the external audio stream based on the user condition.

4. The method of claim 1, wherein a greater weight is applied to either the internal audio stream or the external audio stream based on the user condition.

5. The method of claim 1, wherein a greater weight is applied to the internal audio stream when aggressor signals in the internal audio stream are below a threshold.

6. The method of claim 1, wherein a greater weight is applied to the external audio stream when there are more aggressor signals in the internal audio stream than the external audio stream.

7. The method of claim 1, wherein the respiration rate is determined from a respiration signal by distinguishing the respiration signal from aggressor signals caused by ambient sounds outside of the head worn system.

8. The method of claim 1, wherein the respiration rate is determined based on a first machine learning model that determines a first respiration signal from features extracted from the internal audio stream and a second machine learning model that determines a second respiration signal from features extracted from the external audio stream.

9. The method of claim 1, further comprising:
validating the respiration rate of the user based on an additional audio stream from an additional microphone.

10. The method of claim 1, further comprising:
determining a trust score associated with the respiration rate based on the user condition.

11. The method of claim 1, further comprising:
enhancing the internal audio stream, based on the external audio stream, to generate an enhanced audio stream, wherein the respiration rate is determined from a respiration signal in the enhanced audio stream.

12. A non-transitory computer readable medium storing instructions operable to cause one or more processors to perform operations comprising:
receiving an internal audio stream from an in-ear microphone and an external audio stream from an external microphone of a head worn system;
invoking a machine learning model to determine a respiration signal that measures breathing of a user, the machine learning model determining the respiration signal based on a user condition indicating utilizations of the internal audio stream and the external audio stream; and
determining a respiration rate of the user based on the respiration signal.

13. The non-transitory computer readable medium storing instructions of claim 12, wherein the machine learning model gives greater weight to one of the internal audio stream or the external audio stream, and lesser weight to the other of the internal audio stream or the external audio stream, based on the user condition.

14. The non-transitory computer readable medium storing instructions of claim 12, the operations further comprising:
selecting between either the internal audio stream or the external audio stream based on the user condition.

15. The non-transitory computer readable medium storing instructions of claim 12, the operations further comprising:
validating an aggressor signal, caused by an ambient sound outside of the head worn system, based on an additional audio stream from an additional microphone.

16. The non-transitory computer readable medium storing instructions of claim 12, the operations further comprising:
determining a trust score associated with the respiration rate based on the user condition.

17. A non-transitory computer readable medium storing instructions operable to cause one or more processors to perform operations comprising:
receiving an input indicating a user condition;
receiving an internal audio stream from an in-ear microphone and an external audio stream from an external microphone of a head worn system; and
determining a respiration rate of a user based on the internal audio stream, the external audio stream, and the input indicating the user condition.

18. The non-transitory computer readable medium storing instructions of claim 17,
wherein the respiration rate is updated periodically.

19. The non-transitory computer readable medium storing instructions of claim 17, the operations further comprising:
invoking a machine learning model to determine the respiration rate, wherein the machine learning model is run by a system including at least one of the in-ear microphone or the external microphone.

20. The non-transitory computer readable medium storing instructions of claim 17, the operations further comprising:
invoking a machine learning model to determine the respiration rate, wherein the machine learning model is run by a companion device in communication with at least one of the in-ear microphone or the external microphone.
